(19) United States
(12) Patent Application Publication
(10) Pub. No.: US 2013/0078268 A1
(43) Pub. Date: Mar. 28, 2013

(54) VACCINE COMPOSITIONS AND METHODS OF USE THEREOF

(71) Applicants: Igor Anatolievich Pomytkin, Moscow (RU); Valentin Antonovich Vinogradov, Moscow (RU); Anton Sergeevich Chernopyatko, Moscow (RU)

(72) Inventors: Igor Anatolievich Pomytkin, Moscow (RU); Valentin Antonovich Vinogradov, Moscow (RU); Anton Sergeevich Chernopyatko, Moscow (RU)

(21) Appl. No.: 13/577,662

(22) PCT Filed: Feb. 9, 2010
(86) PCT No.: PCT/RU2010/000050

§ 371 Date: Aug. 8, 2012

(51) Int. Cl. A61K 39/00

(52) U.S. Cl. CPC A61K 39/0005; B82Y 5/00
USPC 424/185.1; 530/327; 977/773; 977/917

(57) ABSTRACT

The present invention relates to vaccine composition and methods for eliciting a protective immune response in animals against an endogenous peptide of the formula pGlu-Pro-Pro-Gly-Gly-Ser-Lys-Val-Ile-Leu-Phe, the composition comprising an immunizing effective amount of said peptide or conjugates thereof. Further, the invention relates to methods of treating cancer, cardiovascular diseases, and increasing life span in an animal, the methods comprising a step of administering to the animal of the vaccine composition of the invention.

### FIELD OF THE INVENTION

The invention relates to vaccine compositions for raising a protective immune response in an animal, including a human being, to the undecapeptide known from the art as hydra head activator.

### BACKGROUND OF THE INVENTION

An undecapeptide having an amino acid sequence of pGlu-Pro-Pro-Gly-Gly-Ser-Lys-Val-Ile-Leu-Phe, wherein pGlu denotes pyroglutamic acid, was originally isolated from the freshwater coelenterate hydra and subsequently found in animals, including humans. The undecapeptide plays a role in normal tissue morphogenesis in animals from coelenterates to humans. The undecapeptide is known in the art as the neuropeptide head activator. At cellular level, the undecapeptide acts as the potent mitogen in G2-mitosis transition and promotes proliferation of different types of cells. Schaller H C, Bodenmuller H, PNAS, 1981, 78(11): 7000-7004. Bodenmuller H, Schaller H C. Nature, 1981, 293:579-580. Schaller H C et al., EMBO J. 1989, 8(11):3311-3318.

Frequently, the endogenous neuropeptide head activator (NHA) is involved in the development of pathological conditions and disorders. Pathologically high levels of the undecapeptide were found in the blood of patients with tumors in peripheral locations, especially in tumors of gastrointestinal tract and/or of neuroendocrine origin. The strong correlation between tumorigenesis and elevated HHA levels was identified. The complete tumor removal resulted in decrease of HHA levels in blood to normal values, whereas incomplete tumor resection was accompanied by a marked decrease of the undecapeptide in blood. Schaller H C et al., J Neurooncol, 1988, 6:251-258, Winnikes M et al., Eur J. Cancer 1992, 28(2-3): 421-4. Elevated HHA levels in blood were shown to induce undesirable proliferation of non-tumor cells, e.g. development of the myocardial hypertrophy. Fedoseev V A et al., Biull Eksp Biol Med 1993, 115(3):307-9: 1993, 116(9):316-8. Thus, there is a need to protect animals, including humans being, from pathologically elevated levels of the endogenous neuropeptide head activator for the preventing or treating hyperproliferative disorders and increasing life span in animals.

U.S. Pat. No. 4,457,917 discloses the undecapeptide having amino acid sequence of pGlu-Pro-Pro-Gly-Gly-Ser-Lys-Val-Ile-Leu-Phe and pharmaceutical compositions comprising this undecapeptide for cell-growth stimulating action. However, no published or disclosed in the art related to any vaccine composition for eliciting protective immune response in animals against endogenous neuropeptide head activator comprising the undecapeptide or its immunogenic conjugates for the purpose of the prevention or treatment of disorders associated with pathologically elevated levels of this undecapeptide.

Non-obviously from the art, we found that vaccination of animals against the endogenous NHA increases its life span of animals, e.g. aged or tumor-bearing animals. The desired protective immune response against the endogenous NHA can be achieved by immunization with the undecapeptide itself, immunogenic conjugates, analogs, or low molecular weight derivatives thereof optionally taken in combination with suitable adjuvant. The optimal immunization can be achieved with the NHA conjugates, wherein the immunogenic conjugate consists of the undecapeptide of formula pGlu-Pro-Pro-Gly-Gly-Ser-Lys-Val-Ile-Leu-Phe covalently linked to an immunogenic carrier or adjuvant.

It is an object of the present invention to provide vaccine compositions for eliciting the protective immune response in animals against endogenous neuropeptide head activator, the composition comprising an immunising effective amount of a peptide of the formula pGlu-Pro-Pro-Gly-Gly-Ser-Lys-Val-Ile-Leu-Phe (SEQ ID NO: 1) or conjugates thereof.

### DETAILED DESCRIPTION OF THE INVENTION

The present invention provides a vaccine composition for eliciting a protective immune response in an animal, the composition comprising an immunizing effective amount of a peptide having a sequence pGlu-Pro-Pro-Gly-Gly-Ser-Lys-Val-Ile-Leu-Phe (SEQ ID NO: 1) or a conjugate thereof, and a pharmaceutically acceptable carrier or adjuvant.

As used herein, the term “vaccine composition” refers to an immunogenic composition which, when administered to an animal, elicits protective immunity against an antigen, in this case the endogenous peptide having the sequence SEQ ID NO: 1.

As used herein, the term “immunizing effective amount” refers to the amount of the peptide having the sequence SEQ ID NO: 1 or a conjugate thereof that, when administered to an animal, is sufficient to elicit protective immune response against an antigen, in this case the endogenous peptide of the sequence SEQ ID NO: 1.

As used herein, the term “protective immune response” refers to eliciting immune response in an animal against the endogenous peptide of SEQ ID NO: 1 that prevents or ameliorates undesirable action of the endogenous peptide or reduces at least one symptom thereof. The level of the immune response can be measured by methods known in the art or as described in the Examples (e.g., by measuring antibody titer).

In some preferred embodiment of the present invention, the peptide of the sequence SEQ ID NO: 1 is used in combination with a suitable adjuvant to enhance the immune response against the endogenous peptide of the sequence SEQ ID NO: 1.

In some preferred embodiment of the present invention, the peptide of the sequence SEQ ID NO: 1 is used as a conjugate with a suitable immunogenic carrier to enhance the immune response against the endogenous peptide of the sequence SEQ ID NO: 1.

As used herein, the term “conjugate” refers to a compound formed by covalent binding of two parts to form a single structure, wherein the first part is the peptide of the amino acid sequence SEQ ID NO: 1 and the second part is an immunogenic carrier. The binding can be made by a covalent chemical bond between the molecules or by use of a linking group, nonexclusively including diaminoalkanes and one or more amino acids, one of which provides a free sulfhydryl, carboxyl, amino or other group for conjugation to the carrier. The preparation of conjugates is described in Hermanson G T. Bioconjugate techniques, second ed., 2008. The carrier protein may be conjugated by one skilled in the art using a variety of chemistries (MBS, EDC and activated EDC), the choice depending on the sequence of the peptide and the position of the peptide in the original protein sequence. Conjugates of the present invention can be prepared by conventional methods by one with ordinary skill in the art. Preferably, it is recommended to conjugate the carrier protein at the free epsilon-amino group of Lys or C-terminus of the peptide SEQ ID NO: 1.

As used herein, the term “immunogenic carrier” refers to an immunogenic substance which, when conjugated to an antigen and administered to an animal, will induce a protective immune response in the animal and elicit the production of antibodies that bind specifically to the antigen, in this case to the endogenous peptide having an amino acid sequence SEQ ID NO: 1. The immunogenic substances include, without limitation, proteins, glycoproteins, complex polyamino-polysaccharides, particles, and nucleic acids that are recognized as foreign and thereby elicit an immunologic response from the host. Nonexclusive examples of immunogenic carriers include bovine serum albumin, keyhole limpet hemocyanin, egg ovalbumin, albumine, and bovine gamma-globulin. In addition, synthetic polypeptides, for example poly(L-lysine), can be employed as carriers. Further reference for the state-of-the-art concerning conventional immunogenic carrier materials and techniques for coupling haptens thereto may be had to the following: Parker, Radioimmunoassay of Biologically Active Compounds, Prentice-Hall (Englewood Cliffs, N.J. USA, 1976); Butler, J. Immunol. Meth. 7:1-24(1974); Weinryb and Schroff, Drug Metab Rev. b 10:271-283(1975); Broughton and Strong, Clin. Chem. 22:726-732(1976); and Playfair et al, Br. Med. Bull. 30:24-31(1974).

Nonexclusive examples of immunogenic carriers are immunogenic protein carriers, lipopeptides, glycolipids, lipoproteins, and molecules capable to stimulate Toll-like receptors, e.g. TLR1, TLR2, TLR3, TLR4, TLR5, TLR6, TLR7, TLR8, TLR9, TLR10, and TLR11.

In some preferred embodiments of the present invention, the conjugate is a compound formed by covalent binding of the free epsilon-amino group of lysine of the peptide of SEQ ID NO: 1 and available carboxylic groups of the immunogenic protein carriers, e.g. keyhole limpet hemocyanin (KLH), bovine serum albumin (BSA), and ovalbumin (OVA).

In one preferred embodiment of the present invention, the conjugate is pGlu-Pro-Pro-Gly-Gly-Ser-Lys(KLH)-Val-Ile-Leu-Phe.

In another preferred embodiment of the present invention, the conjugate is pGlu-Pro-Pro-Gly-Gly-Ser-Lys(BSA)-Val-Ile-Leu-Phe.

In another preferred embodiment of the present invention, the conjugate is pGlu-Pro-Pro-Gly-Gly-Ser-Lys(OVA)-Val-Ile-Leu-Phe.

In some preferred embodiments of the present invention, the conjugate is a compound formed by covalent binding of the free epsilon-amino group of lysine of the peptide of SEQ ID NO: 1 and C-terminus of lipopeptides, e.g. the tripalmitoyl-S-glyceryl-cysteine (Pam3Cys), dipalmitoyl-S-glyceryl-cysteine (Pam2Cys), and N-acetyl-dipalmitoyl-S-glyceryl-cysteine (N-Ac-Pam2Cys), wherein “dipalmitoyl-S-glyceryl-cysteine” is a compound of formula (I)

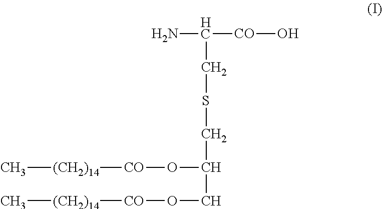

“N-acetyl-dipalmitoyl-S-glyceryl-cysteine” is a compound of formula (II)

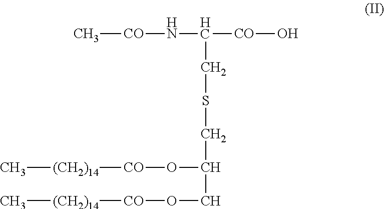

“tripalmitoyl-S-glyceryl-cysteine” is a compound of formula (III)

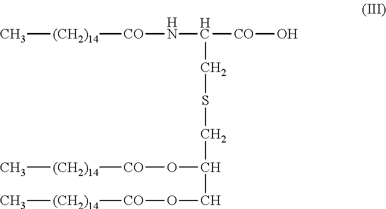

In one preferred embodiment of the present invention, the conjugate is pGlu-Pro-Pro-Gly-Gly-Ser-Lys(Pam3Cys)-Val-Ile-Leu-Phe.

In another preferred embodiment of the present invention, the conjugate is pGlu-Pro-Pro-Gly-Gly-Ser-Lys(Pam2Cys)-Val-Ile-Leu-Phe.

In another preferred embodiment of the present invention, the conjugate is pGlu-Pro-Pro-Gly-Gly-Ser-Lys(N-Ac-Pam2Cys)-Val-Ile-Leu-Phe.

The lipopeptide conjugates of the invention may be prepared by solid phase synthesis well-known from the art for example, as it disclosed by Metzger et al., Synthesis of novel immunologically active tripalmitroyl-S-glycerylcysteinyl lipopeptides as useful intermediates for immunogen preparations. Int. J. Pept. Prot. Res. 1991, 37: 46-57.

As used herein, the term “pharmaceutically acceptable carrier” refers to any ingredient having no therapeutic activity and being nontoxic and thus suitable as carrier. Nonexclusive suitable carriers will include any of the carriers commonly used in pharmaceutical products, such as, for example, water for injections, microcrystalline cellulose, lactose and starch.

As used herein, the term “pharmaceutically acceptable adjuvant” refers to a substance that enhances, augments or potentiates the host's immune response to an antigen, in this case the endogenous peptide having the amino acid sequence SEQ ID NO: 1. Such adjuvants include, but are not limited to, mineral oil emulsions; aluminum hydroxide; cytokines, such as interleukins (IL-1, IL-2, etc.), macrophage colony stimulating factor (M-CSF), tumor necrosis factor (TNF), beta chemokines (MIP, 1-alpha, 1-beta Rantes, etc.); detoxified mutants of a bacterial ADP-ribosylating toxin such as a cholera toxin (CT), a pertussis toxin (PT), or an E. coli heat-labile toxin (LT); polymers, co-polymers such as polyoxyethylene-polyoxypropylene copolymers, including block co-polymers; polymer P1005; Freund's complete adjuvant (for animals); Freund's incomplete adjuvant; sorbitan monooleate; squalene; CRL-8300 adjuvant; alum; QS 21, muramyl dipeptide; CpG oligonucleotide motifs and combinations of CpG oligonucleotide motifs; trehalose; bacterial extracts, including mycobacterial extracts; detoxified endotoxins; membrane lipids; or combinations thereof. A suitable adjuvant may be selected by one ordinary skilled in the art with the use of the guideline in the field of adjuvants; Hackett C J, Harn D A. Vaccine adjuvants: immunological and clinical principles. 2006.

In one preferred embodiment of the present invention, the adjuvant is calcium phosphate nanoparticles.

In some preferred embodiments of the present invention, the content of the peptide of the formula SEQ ID NO: 1 in vaccine compositions of the present invention is from 0.001 to 20 wt %.

In some preferred embodiments of the present invention, the content of the conjugate of the peptide of the formula SEQ ID NO: 1 in vaccine compositions of the present invention is from 0.001 to 20 wt. %.

The compositions of the present invention can comprise optional ingredients. Such optional ingredients generally are used individually at levels from about 0.0005% to about 10.0%, preferably from about 0.005% to about 1.0% by weight of the composition.

Examples of suitable optional ingredients include, but are not limited to, solvents, buffers, emulsifiers, and preservatives.

Further, the present invention provides the use of a peptide of the formula pGlu-Pro-Pro-Gly-Gly-Ser-Lys-Val-Ile-Leu-Phe (SEQ ID NO: 1) or a conjugate thereof for manufacturing the vaccine composition of the present invention for eliciting a protective immune response in an animal against the endogenous peptide of the formula SEQ ID NO: 1.

The vaccine compositions of the invention may be prepared by standard techniques well known to those skilled in the art. Such procedures include, but are not limited to, mixing the peptide of the formula SEQ ID NO: 1 or a conjugate thereof with other ingredients of the composition in conventional manner. Accordingly, the peptide, or conjugates thereof, can be formulated as a vaccine composition using adjuvants, pharmaceutically-acceptable carriers, excipients, diluents, auxiliary agents or other ingredients routinely provided in vaccine compositions. Such formulations are readily determined by one of ordinary skill in the art and include formulations for immediate release and for sustained release, e.g., microencapsulation. The present vaccine compositions can be administered by any convenient route including subcutaneous, intramuscular, oral, oromucosal, or other parenteral or internal route. Similarly the vaccines can be administered as a single dose or divided into multiple doses for administration. Immunization schedules are readily determined by the one ordinary skilled in the art.

Further, the present invention provides a method for eliciting protective immunity in an animal against an endogenous peptide of the formula pGlu-Pro-Pro-Gly-Gly-Ser-Lys-Val-Ile-Leu-Phe (SEQ ID NO: 1), the method comprising a step of administering to the animal an immunizing effective amount of the vaccine composition of the present invention.

In practicing the methods of the present invention, an immunizing effective amount of the vaccine compositions of the present invention may be administered by a variety of routes including, but are not limiting to, injections, e.g. subcutaneous, intramuscular, or intravenous; topical application to the mucosal epithelia; intranasal, and oral administration. Effective amounts of the vaccine composition of present invention may vary on animal species and route of administration and is expected to vary from about 0.001 mg/kg body weight per day to about 100 mg//kg per day. Preferred amounts may be determined by one skilled in the art, e. g. by measuring antibody titer.

As used herein, the term “animal” refers to humans (male or female), companion animals (e.g., dogs, cats and horses), food-source animals, zoo animals, marine animals, birds and other animal species.

Since the endogenous peptide SEQ ID NO: 1 is ubiquitously distributed in animals from the freshwater coelenterate hydra to humans and the peptide is the universal mitogen for different types of cells and elevated levels of the endogenous peptide is associated with enhanced call growth and proliferation, the vaccine compositions of the invention may be used for the prevention or treatment of a broad spectrum of diseases and disorders wherein the proliferation of cells is pathologically changed. Such diseases and disorders include, but are not limited to, neoplasms (cancer), musculoskeletal diseases, digestive system diseases, respiratory tract diseases, otorhinolaryngologic diseases, nervous system, diseases, eye diseases, male urogenital diseases, female urogenital diseases, cardiovascular diseases, hemic and lymphatic diseases, congenital, hereditary, and neonatal diseases and abnormalities, skin and connective tissue diseases, nutritional and metabolic diseases, endocrine system diseases, immune system diseases, animal diseases, pathological conditions, signs and symptoms, behavior and mental disorders.

Nonexclusive examples of neoplasms include adenoma, adenomatous polyposis coli, angiofibroma, arachnoid cysts, astrocytoma, basal cell nevus syndrome, bone neoplasms, bowen's disease, breast cyst, breast neoplasms (breast cancer), breast neoplasms, male, burkitt lymphoma, carcinoid tumor, carcinoma, carcinoma, basal cell, carcinoma, merkel cell, cementoma, chalazion, choledochal cyst, chondroma, chondrosarcoma, chordoma, craniopharyngioma, cysts, dentigerous cyst, dermoid cyst, digestive system neoplasms, ear neoplasms, endocrine gland neoplasms, endometrial neoplasms, ependymoma, epidermal cyst, Epstein-Barr virus Infections, eye neoplasms, fibromatosis, juvenile hyaline, gastrointestinal neoplasms, gastrointestinal stromal tumors, genital neoplasms, glioblastoma, glioma, hamartoma, hamartoma syndrome, multiple head and neck neoplasms, hemangioma, cavernous, hemangiosarcoma, histiocytoma, benign fibrous, histiocytoma, malignant fibrous, Hodgkin disease, Hutchinson's melanotic freckle, hydatidiform mole, insulinoma, intestinal neoplasms, Krukenberg tumor, Lambert-Eaton myasthenic syndrome, laryngeal neoplasms, leiomyoma, leiomyosarcoma, leukemia, lipoma, lung neoplasms, lymphangioleiomyomatosis, lymphangioma, lymphoma, Non-Hodgkin lymphoma, mediastinal cyst, medulloblastoma, melanoma, meningioma, mesothelioma, mouth neoplasms, multiple myeloma, myoma, myxoma, Nelson syndrome, neoplasm metastasis, nervous system neoplasms, neurilemmoma, neuroblastoma, neuroendocrine tumors, neurofibromatoses, neuroma, odontogenic tumors, osteosarcoma, otorhinolaryngologic neoplasms, ovarian cysts, ovarian neoplasms, Paget's disease, pancreatic neoplasms, papilloma, paraganglioma, paraneoplastic syndromes, Peutz-Jeghers syndrome, pheochromocytoma, pilonidal sinus, polycystic ovary syndrome, popliteal cyst, precancerous conditions, prostatic neoplasms (prostate cancer), proteus syndrome, pseudomyxoma peritonei, ranula, rectal neoplasms, respiratory tract neoplasms, retinoblastoma, rhabdoid tumor, rhabdomyosarcoma, sarcoma, skin neoplasms, Sturge-Weber syndrome, Tarlov cysts, teratoma, testicular neoplasms, thoracic neoplasms, thymoma, thyroid neoplasms, thyroid nodule, tonsillar neoplasms, trophoblastic neoplasms, tuberous sclerosis, tumor virus infections, urinary bladder neoplasms, urologic neoplasms, uterine cervical dysplasia, uterine cervical neoplasms, Waldenstrom macroglobulinemia, warts, wilms tumor, vulvar neoplasms, xeroderma pigmentosum, and Zollinger-Ellison syndrome.

As used herein, “cardiovascular disease” has the meaning commonly used in the field, and includes, but is not limited to, the following diseases or conditions: thromboembolic disorders, including arterial cardiovascular thromboembolic disorders, venous cardiovascular thromboembolic disorders, and thromboembolic disorders in the chambers of the heart; ahtherosclerosis; restensosis; peripheral arterial disease; coronary bypass grafting surgery; carotid artery disease; arteritis; myocarditis; cardiovascular inflammation; vascular inflammation; coronary heart disease (CHD); unstable angina (UA); unstable refractory angina; stable angina (SA); chronic stable angina; acute coronary syndrome (ACS); first or recurrent myocardial infarction; acutemyocardial infarction (AMI); myocardial infarction; non-Q wave myocardial infarction; non-STE myocardial infarction; coronary artery disease; cardiac ischemia; ischemia; ischemic sudden death; transient ischemic attack; stroke; atherosclerosis; peripheral occlusive arterial disease; venous thrombosis; deep vein thrombosis; thrombophlebitis; arterial embolism; coronary arterial thrombosis; cerebral arterial thrombosis; cerebral embolism; kidney embolism; pulmonary embolism; thrombosis resulting from (a) prosthetic valves or other implants, (b) indwelling catheters, (e) stents, (d) cardiopulmonary bypass, (e) hemodialysis, or (f) other procedures in which blood is exposed to an artificial surface that promotes thrombosis; thrombosis resulting from atherosclerosis, surgery or surgical complications, prolonged immobilization, arterial fibrillation, congenital thrombophilia, cancer, diabetes, effects of medications or hormones, and complications of pregnancy; cardiac arrhytmias including supraventricular arrhythmias, atrial arrhythmias, atrial flutter, atrial fibrillation; other diseases listed in Heart Disease: A Textbook of Cardiovascular Medicine, 2 Volume Set, 6th Edition, 2001, Eugene Braunwald, Douglas P. Zipes, Peter Libby, Douglas D. Zipes.

The present invention provides a method for treating or preventing cancer in an animal, the method comprising a step of administering to the animal an immunizing effective amount of the vaccine composition of the present invention.

The present invention provides a method for increasing life span in an animal, the method comprising a step of administering to the animal an immunizing effective amount of the vaccine composition of the present invention.

The present invention provides a method for treating a cardiovascular disease, the method comprising a step of administering to the animal, an immunizing effective amount of the vaccine composition of the present invention.

The following examples are presented to demonstrate the invention. The examples are illustrative only and are not intended to limit the scope of the invention in any way.

### EXAMPLE 1

This example shows the synthesis the peptide SEQ ID NO: 1 and conjugates thereof with immunogenic protein carriers.

The peptide of SEQ ID NO: 1 was synthesized by the standard solid phase technique. Peptide purity was >95% by HPLC.

Conjugation of the peptide of SEQ ID NO: 1 to an immunogenic protein carrier was performed by two different conventional methods. The first method involved the reaction of the free C-terminal acid of the peptide SEQ ID NO: 1 to a lysine residue of the carrier. Briefly, 5 mg of the peptide SEQ ID NO: 1 was dissolved in 1 ml of water and 20 mg of EDC (1-ethyl-3-(3-dimethylaminopropyl) carbodiimide hydrochloride) was added to the stirred peptide solution and adjusted the pH to 4.5 using 0.1 M hydrochloric acid solution. The mixture was incubated for 10 min at room temperature. 100 mg of the immunogenic protein carrier was dissolved in 2 ml of water and added to the activated peptide solution. The resulted mixture was stirred for 2 hours at room temperature and dialyzed twice for 4 hours against 5 liters of PBS buffer. After lyophilization, it provides the conjugate of the peptide of SEQ ID NO: 1, e.g. pGlu-Pro-Pro-Gly-Gly-Ser-Lys-Val-Ile-Leu-Phe(KLA) conjugate 1), or pGlu-Pro-Pro-Gly-Gly-Ser-Lys-Val-Ile-Leu-Phe(BSA) (conjugate 2), or pGlu-Pro-Pro-Gly-Gly-Ser-Lys-Val-Ile-Leu-Phe(OVA) (conjugate 3), when keyhole limpet hemocyanin (KLH), bovine serum albumin (BSA), or ovalbumin (OVA) were used as the immunogenic carrier. The second method involved the reaction of the free epsilon-amino group of the peptide SEQ ID NO: 1 to lysine group of an immunogenic protein carrier. Briefly, 100 mg of the immunogenic protein carrier was dissolved in 2 ml of water for 4 hours. The solution was dialyzed overnight against 2 liters of 0.1 M sodium phosphate pH 7.8 to remove contaminants. Aggregates were removed on spin microcentifuge. 5 mg of the peptide SEQ ID NO: 1 was added to 100 ml of the immunogenic protein carrier solution, followed by glutaraldehyde to 0.1% final, and then the pH was adjusted to 7.8 by sodium hydroxide. The mixture was incubated under gentle rotating for 12 hours at 4 degrees. The resulted mixture was stirred for 2 hours at room temperature and dialyzed twice for 4 hours against 5 liters of PBS buffer. After lyophilization, it provides the conjugate of the peptide of SEQ ID NO: 1, e.g. pGlu-Pro-Pro-Gly-Gly-Ser-Lys(KLH)-Val-Ile-Leu-Phe (conjugate 4), pGlu-Pro-Pro-Gly-Ser-Lys(BSA)-Val-Ile-Leu-Phe (conjugate 5), or pGlu-Pro-Pro-Gly-Gly-Ser-Lys(OVA)-Val-Ile-Leu-Phe (conjugate 6), when keyhole limpet hemocyanin (KLH), bovine serum albumin (BSA), or ovalbumin (OVA) were used as the immunogenic carrier. Protein content of dialyzed conjugates and control was determined by the bicinchoninic acid assay (Pierce). Quantitation of peptide incorporation into the conjugate was determined by amino acid analysis as previously described. Shuler K R et al., J Immunol Methods 156:137 (1992).

### EXAMPLE 2

This example shows the synthesis the peptide SEQ ID NO: 1 and conjugates thereof with lipopeptides.

Conjugates of the peptide of SEQ ID NO: 1 with the tripalmitoyl-S-glyceryl-cysteine (Pam3Cys), dipalmitoyl-S-glyceryl-cysteine (Pam2Cys), or N-acetyl-dipalmitoyl-S-glyceryl-cysteine (N-Ac-Pam2Cys) were prepared by solid phase methods similar to that was described in Metzger et al., Synthesis of novel immunologically active tripalmitroyl-S-glycerylcysteinyl lipopeptides as useful intermediates for immunogen preparations. Int. J. Pept. Prot. Res. 1991, 37: 46-57. Briefly, a commercially available Pam3Cys-OH, Pam2Cys-OH, or synthetic NAcPam2Cys-OH were coupled to immobilized peptide SEQ ID NO: 1 prepared through standard Fmoc-SPPS using HCTU as coupling reagent. HPLC purification over a CN-column yielded pure hydrophobic lipopeptide conjugate. After lyophilization, it provides the conjugate of the peptide of SEQ ID NO: 1, e.g. pGlu-Pro-Pro-Gly-Gly-Ser-Lys(Pam3Cys)-Val-Ile-Leu-Phe (conjugate 7), pGlu-Pro-Pro-Gly-Gly-Ser-Lys(Pam2Cys)-Val-Ile-Leu-Phe (conjugate 8), and pGlu-Pro-Pro-Gly-Gly-Ser-Lys(NAcPam2Cys)-Val-Ile-Leu-Phe (conjugate 9), when Pam3Cys, Pam2Cys, or NAcPam2Cys were used as the immunogenic lipopeptide carrier.

### EXAMPLE 3

This example shows the use of peptide SEQ ID NO: 1 or conjugates thereof for preparation of vaccine compositions.

The 100 mg of the lyophilized peptide of SEQ ID NO: 1 or the conjugates 1 through 9 thereof were mixed with 100 ml of soybean oil and 100 ml of a mixture of 1-oleoyl monoglyceride and oleic acid (1:1). The mixture was sonicated and then 1 L of 0.1 M Tris buffer and 20 ml of 4 M NaOH were added. After the final sonication for 1 min the emulsion was used for the immunization.

TABLE 1

| Ingredient | Content, wt. % |
|---|---|
| Lyophilized peptide SEQ ID NO: 1 or the conjugate | 0.01-10 |
| thereof | |
| Soybean oil | 7 |
| 1-Oleoyl monoglyceride | 3.5 |
| Oleic acid | 3.5 |
| Tris buffer | 9 |
| NaOH | 0.25 |
| Water for injections | to 100 |

### EXAMPLE 4

This example shows the method for eliciting protective immunity in animals against endogenous peptide of SEQ ID NO: 1 with vaccine composition comprising the peptide of SEQ ID NO: 1 and calcium phosphate nanoparticles as the adjuvant.

Four male mice (8-week-old, C57B1) were given, three subcutaneous injections of vaccine composition comprising 100 μg of the peptide SEQ ID NO: 1 and 100 μg of calcium phosphate nanoparticles at 3-week intervals. As controls, an additional four mice received the vehicle. Two weeks after the last injection, the titer of antibody to the peptide of SEQ ID NO: 1 was assessed by the peptide-based enzyme-linked immunosorbent assay (ELISA). Samples were run in triplicate on ELISA plates coated with the peptide of the SEQ ID NO: 1, which was used as the immunogen. The peptide was bound in solid phase on high binding 96 well plates. Serum was diluted 1:500 and then serially diluted to a final dilution of 1:64,000. Detection of the antibody was obtained using an HRP conjugated secondary antibody (1:5,000) and TMB micro well peroxidase substrate. The geometric mean titer of anti-SEQ ID NO: 1 antibodies in serum of mice immunized with the vaccine composition was found to be from about 70,000 for and not detected in control mice, wherein the individual titer was defined, as the dilution giving more than 2 times the optical density of the corresponding dilution of pre-bleed serum.

### EXAMPLE 5

This example shows the method tor eliciting protective immunity in animals against endogenous peptide of SEQ ID NO: 1 with vaccine compositions.

Four male mice (8-week-old, C57B1) were given three intraperitoneal injections of vaccine composition of example 3 at 3-week intervals. As controls, an additional four mice received the vehicle. Two weeks after the last injection, the titer of antibody to the peptide of SEQ ID NO: 1 was assessed by the peptide-based enzyme-linked immunosorbent assay (ELISA) as described in the example 4. The geometric mean titer of anti-SEQ ID NO: 1 antibodies in serum of mice immunised with the vaccine compositions comprising the conjugates 1 through 9 were found to be from about 120,000 to 180,000 for and not detected in control mice. Thus, the administering vaccine compositions of the invention elicited protective immunity in animals against endogenous peptide of SEQ ID NO: 1.

### EXAMPLE 6

This example shows that vaccine composition is useful for treating cancer in animals.

Female BALB/c mice were given three intraperitoneal injections of vaccine composition of example 3 at 3-week intervals. Then, mice were randomized, and one group (n=15) received i.p. saline (control), while second group (n=15) received suspension of 5×10⁵/0.05 ml of HT-29 tumor cells (test). The survival period of the immunised with vaccine composition tumor bearing mice was 27±8 days as compared to 19±6 days in control group (p<0.05). Thus, the immunization with vaccine composition significantly increased survival and life span in tumor-bearing mice.

### EXAMPLE 7

This example shows that vaccine composition is useful for increasing life span in animals under normal ageing.

Female BALB/c mice of 16 weeks of age were given three intraperitoneal injections of vaccine composition of example 3 comprising 100 μg of conjugate of formula pGlu-Pro-Pro-Gly-Gly-Ser-Lys(Pam3Cys)-Val-Ile-Leu-Phe (n=30) or saline (control, n=30) at 3-week intervals. Mean life span of mice immunised with the vaccine composition was 780±38 days as compared to 540±21 days in control mice (p<0.05). Thus, the immunisation with vaccine composition significantly increased life span in animals under normal ageing.

### EXAMPLE 8

This example shows that vaccine composition is useful for treating a cardiovascular disease.

Female BALB/c mice of 16 weeks of age were given three intraperitoneal injections of vaccine composition of example 3 comprising 100 μg of conjugate of formula pGlu-Pro-Pro-Gly-Gly-Ser-Lys(Pam3Cys)-Val-Ile-Leu-Phe (n=10) or saline (control, n=10) at 3-week intervals. Then, mice were given the single injection of the neuropeptide head activator (NHA) to develop a heart hypertrophy as described in Fedoseev et al., Effect of peptide hydra morphogen on the structure of tissue components of rat myocardial layers in the early period of heart hypertrophy development. Biull Eksp Biol Med. 1993, 115(3):307-9. The vaccination with the conjugate of the peptide SEQ ID NO: 1 was found to completely prevent the hypertrophy of muscular and connective tissue components of the myocardium as well as myocardium wall vessels as compared to the control.

1. A vaccine composition for eliciting a protective immune response in an animal, the composition comprising an immunising effective amount of a peptide of the formula pGlu-Pro-Pro-Gly-Gly-Ser-Lys-Val-Ile-Leu-Phe (SEQ ID NO: 1) or a conjugate thereof, and a pharmaceutically acceptable carrier or adjuvant.

2. The vaccine composition of claim 1, wherein the conjugate consists of the peptide of the SEQ ID NO: 1 covalently linked to an immunogenic carrier.

3. The vaccine of claim 2, wherein the immunogenic carrier is selected from the group consisting of an immunogenic protein carrier, lipopeptide, glycolipid, and lipoprotein.

4. The vaccine composition of claim 2, wherein the conjugate consists of the peptide of the SEQ ID NO: 1 covalently linked to lipopeptide selected from the group consisting of dipalmitoyl-S-glyceryl-cysteine (Pam2Cys), N-acetyl-dipalmitoyl-S-glyceryl-cysteine (NAcPam2Cys), and tripalmitoyl-S-glyceryl cysteine (Pam3Cys).

5. The vaccine composition of claim 4, wherein the conjugate is selected from the group consisting of pGlu-Pro-Pro-Gly-Gly-Ser-Lys(Pam2Cys)-Val-Ile-Leu-Phe, pGlu-Pro-Pro-Gly-Gly-Ser-Lys(NAcPam2Cys)-Val-Ile-Leu-Phe, and pGlu-Pro-Pro-Gly-Gly-Ser-Lys(Pam3Cys)-Val-Ile-Leu-Phe.

6. The vaccine composition of claims 1 though 5, wherein the adjuvant is calcium phosphate nanoparticles.

7. Use of a peptide of the formula pGlu-Pro-Pro-Gly-Gly-Ser-Lys-Val-Ile-Leu-Phe (SEQ ID NO: 1) or a conjugate thereof for manufacturing the vaccine composition of claim 1 through 6 for eliciting a protective immune response in an animal against the endogenous peptide of the formula SEQ ID NO: 1.

8. A method for eliciting protective immunity in an animal against an endogenous peptide of the formula pGlu-Pro-Pro-Gly-Gly-Ser-Lys-Val-Ile-Leu-Phe (SEQ ID NO: 1), the method comprising a step of administering to the animal an immunizing effective amount of the vaccine composition of claim 1 through 6.

9. A method for treating or preventing cancer in an animal, the method comprising a step of administering to the animal an immunizing effective amount of the vaccine composition of claim 1 through 6.

10. A method for increasing life span in an animal, the method comprising a step of administering to the animal an immunising effective amount of the vaccine composition of claim 1 through 6.

11. A method for treating a cardiovascular disease, the method comprising a step of administering to the animal an immunising effective amount of the vaccine composition of claim 1 through 6.


SEQUENCE LISTING (1 sequence)

SEQ ID NO: 1 - protein, 11 aa, Homo sapiens
  MISC_FEATURE (1)..(11): Synthetic peptide
XPPGGSKVILF